(19) United States
(12) Patent Application Publication
(10) Pub. No.: US 2010/0042108 A1
(43) Pub. Date: Feb. 18, 2010

(54) METHOD OF CARDIAC SURGERY, AND DEFIBRILLATION ELECTRODE, DEFIBRILLATOR, AND ENDOSCOPE APPARATUS FOR THE SAME

(75) Inventor: Hiroki HIBINO, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/511,481

(22) Filed: Jul. 29, 2009

(30) Foreign Application Priority Data
Jul. 30, 2008 (JP) 2008-197044

(51) Int. Cl. A61B 17/00; A61F 2/06; A61N 1/05; A61N 1/39

(52) U.S. Cl. 606/129; 623/1.23; 607/129; 607/5

(57) ABSTRACT

To provide a method of cardiac surgery which is capable of facilitating the manipulation of devices in a thoracic cavity so as to thereby simplify the surgery, as well as alleviating physical burdens on a patient, and a defibrillation electrode, a defibrillator, and an endoscope apparatus for the same. There is provided a method of cardiac surgery, comprising: using a device which comprises a treatment unit to be inserted into a thoracic cavity for performing treatment of a heart; inserting a distal end of this device between the heart and a pericardium; thereafter piercing through the pericardium with the distal end of the device; and performing treatment of the heart from the outside of the pericardium by using the treatment unit.

### BACKGROUND OF THE INVENTION

1. Field of the Invention

The present invention relates to a method of cardiac surgery, and a defibrillation electrode, a defibrillator, and an endoscope apparatus for the same.

2. Description of Related Art

Conventionally well-known cardiac surgeries include: surgery for setting lead wires of a body implantable heart treatment apparatus in a heart; and coronary artery bypass surgery for connecting between upstream and downstream sides of a coronary artery stenosis site by using a graft. U.S. Pat. No. 6,478,029 discloses a method for coronary artery bypass surgery in which devices are inserted from intercostal spaces into the thoracic cavity so as to thereby connect a graft to a coronary artery without open-chest approaches. U.S. Pat. No. 4,911,603 discloses a tool for arranging electrode portions which are provided on the distal ends of ventricular defibrillation lead wires, along the outer peripheral surface of the pericardium or the epicardium. In the thoracic cavity, lungs and other tissues do exist as well as the heart in a close contact manner with each other, and some tissues may form adhesions. Accordingly, in order to introduce a device to the heart within the thoracic cavity, it is necessary to move the device by separating the closely contacted or adhered tissues and to deflate one of the lungs through single lung ventilation so as to thereby retain a space in the thoracic cavity.

Meanwhile, an endoscope has been employed as a surgery device for low invasive treatment of the inside of the body of a patient. U.S. Pat. No. 5,297,536 and U.S. Pat. No. 5,458,131 disclose methods of intraabdominal surgery in which a device is inserted from the mouth or the anus and moved by piercing through a cavity wall such as the stomach wall or the intestinal wall to thereby introduce the device to a desired position in the abdominal cavity, and then the inside of the abdominal cavity is treated.

### BRIEF SUMMARY OF THE INVENTION

The present invention takes such a situation into consideration with an object of providing a method of cardiac surgery which is capable of facilitating the manipulation of devices in a thoracic cavity so as to thereby simplify the surgery, as well as alleviating physical burdens on a patient, and a defibrillation electrode, a defibrillator, and an endoscope apparatus for the same.

In order to achieve the above object, the present invention provides the following solution.

It is a first aspect of the present invention to provide a method of cardiac surgery comprising, upon a cardiac surgery with use of a device which comprises on its distal end a treatment unit insertable in a thoracic cavity for performing treatment of a heart: inserting the distal end of the device between the heart and a pericardium; thereafter piercing through the pericardium with the distal end of the device; and performing treatment of the heart from the outside of the pericardium by using the treatment unit.

### BRIEF DESCRIPTION OF THE SEVERAL VIEWS OF THE DRAWINGS

FIG. 1 is an overall configuration diagram illustrating a defibrillator comprising defibrillation electrodes according to one embodiment of the present invention.

FIG. 2 is an overall configuration diagram illustrating an endoscope apparatus according to one embodiment of the present invention for use in implantation of the defibrillator of FIG. 1 into a body.

FIG. 3 is a cross sectional view of a bendable portion of an insertion portion of the endoscope apparatus of FIG. 2.

FIG. 4 is a longitudinal sectional view of a channel portion for backward and forward movement of a defibrillation electrode of the bendable portion of the insertion portion of the endoscope apparatus of FIG. 2.

FIG. 5 is a longitudinal sectional view of the channel portion which houses an observation optical system within the bendable portion of the insertion portion of the endoscope apparatus of FIG. 2.

FIG. 6A and FIG. 6B show longitudinal sectional views, wherein FIG. 6A illustrates a state where the defibrillation electrode is housed in the channel of the insertion portion, and FIG. 6B illustrates a state where a part of the defibrillation electrode is sent out from the channel of the insertion portion.

FIG. 7 is an explanatory side view of the operation of the bendable portion of the insertion portion of the endoscope apparatus of FIG. 2.

FIG. 8 illustrates the defibrillation electrode in a state where the electrode portion is set outside the pericardium.

FIG. 9 is an explanatory diagram of a step of inserting a guide wire in a layout method for setting the defibrillation electrode of the defibrillator of FIG. 1 in the body of a patient.

FIG. 10 is an explanatory diagram of a step of inserting a sheath along the guide wire that has been inserted in FIG. 9.

FIG. 11 is an explanatory diagram of a step of inserting the insertion portion of the endoscope apparatus through the sheath that has been inserted in FIG. 10.

FIG. 12 illustrates a state where the pericardium is pierced with the defibrillation electrode projecting from the distal end of the insertion portion of the endoscope apparatus that has been inserted in FIG. 11.

FIG. 13 illustrates a state where the electrode portion of the defibrillation electrode that has been inserted in FIG. 12, is set outside the pericardium facing the left ventricle.

FIG. 14 illustrates a state where two electrode portions of the defibrillation electrodes are set outside the pericardium facing the left ventricle and outside the pericardium facing the right ventricle.

FIG. 15 illustrates a state where the defibrillator mainbody connected to the defibrillation electrodes is implanted in the body of the patient.

FIG. 16A and FIG. 16B illustrate a modified example of the defibrillation electrode of FIG. 1, showing the defibrillation electrode comprising a different stopper which has a sharp portion on the distal end: (FIG. 16A) before piercing the pericardium; (FIG. 16B) after piercing through the pericardium.

FIG. 17A and FIG. 17B show the insertion portion of the endoscope apparatus of FIG. 2, wherein FIG. 17A illustrates the operation of the bendable portion, and FIG. 17B illustrates a state where an armored tube is pushed out from the channel.

FIG. 18 illustrates a state where the endoscope apparatus of FIG. 17 is used to project the defibrillation electrode to the interior of the pericardium from the armored tube that has been once inserted to the outside of the pericardium.

FIG. 19 illustrates a state where, continued from the state of FIG. 18, the stopper is fixed to the inner surface of the pericardium, and the coiled electrode portion is sent out from the armored tube.

FIG. 20 illustrates a state where, continued from the state of FIG. 19, the armored tube and the insertion portion are withdrawn.

FIG. 21 illustrates a modified example where a pacing electrode portion is attached to the stopper provided on the defibrillation electrode of FIG. 1.

FIG. 22 illustrates a modified example of the electrode portion provided on the defibrillation electrode of FIG. 1.

FIG. 23 illustrates a modified example of the insertion portion of the endoscope apparatus of FIG. 2, seen from the distal side.

FIG. 24 is a longitudinal sectional view of the bendable portion of the insertion portion of the endoscope apparatus of FIG. 23.

FIG. 25 illustrates a modified example of the layout method of FIG. 20 showing a state where the electrode portion is placed inside the pericardium.

FIG. 26 illustrates a modified example of FIG. 15 showing a state where a defibrillator comprising a thinner and more compact defibrillator mainbody is implanted in the body of the patient.

FIG. 27 illustrates a modified example of the layout method of FIG. 18 showing a state where the defibrillation electrode is projected to the interior of the pericardium from the insertion portion which is placed outside the pericardium.

FIG. 28 illustrates a modified example of the insertion portion of the endoscope apparatus for use in the layout method of FIG. 27.

FIG. 29 illustrates a modified example of the layout method of the defibrillation electrode showing a state where the distal portion of the sheath is inserted to the outside of the pericardium.

FIG. 30 illustrates an example of a compressive deformation method of a lung.

FIG. 31 illustrates a modified example of the pathway for inserting the sheath into the pericardium, which is a transvenous insertion pathway from the right auricle into the pericardium.

FIG. 32 illustrates another modified example of the pathway for inserting the sheath into the pericardium, which is a transvenous insertion pathway from a coronary vein into the pericardium.

FIG. 33 illustrates yet another modified example of the pathway for inserting the sheath into the pericardium, which is a transvenous insertion pathway from the inferior vena cava outside the pericardium into the pericardium.

FIG. 34 is an explanatory diagram of a method for performing a coronary artery bypass surgery by using the method of cardiac surgery of the present invention.

### DETAILED DESCRIPTION OF THE INVENTION

Hereunder is a description of a method of cardiac surgery according to one embodiment of the present invention, and a defibrillation electrode 1, a defibrillator 2, and an endoscope apparatus 3 for the same, with reference to drawings.

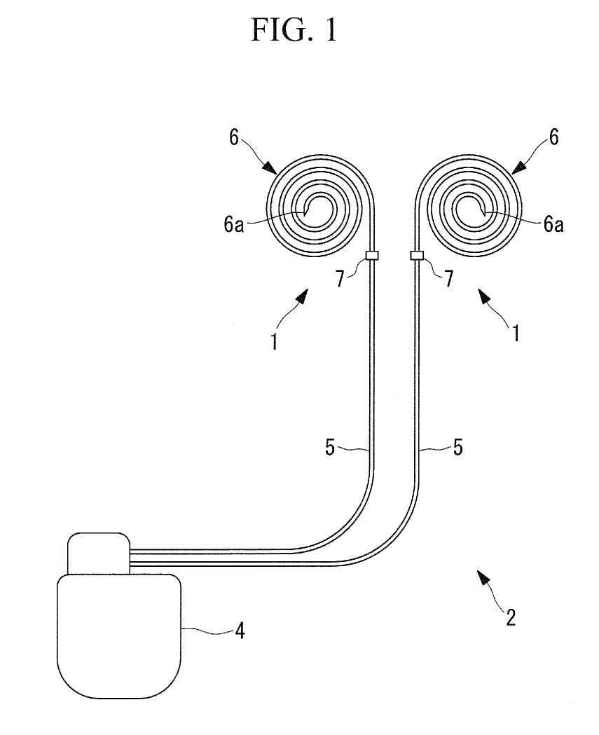

As shown in FIG. 1, the defibrillator 2 according to this embodiment is a body implantable defibrillator 2 comprising a pair of defibrillation electrodes 1 and a defibrillator mainbody 4 for applying a defibrillation voltage between these defibrillation electrodes 1.

As shown in FIG. 1, the defibrillation electrode 1 according to this embodiment comprises a lead wire 5 extending from the defibrillator mainbody 4, a coiled electrode portion 6 provided on the distal end of the lead wire 5, and a stopper 7 provided on the lead wire 5 in a vicinity of the electrode portion 6.

An insulating coating (not shown) is applied on the lead wire 5 from the defibrillator mainbody 4 to the electrode portion 6.

The electrode portion 6 made of a bare wire which constitutes a part of the lead wire 5, is formed in a coiled shape in a free state as shown in FIG. 1, and is extendable into an approximate linear shape by an external force.

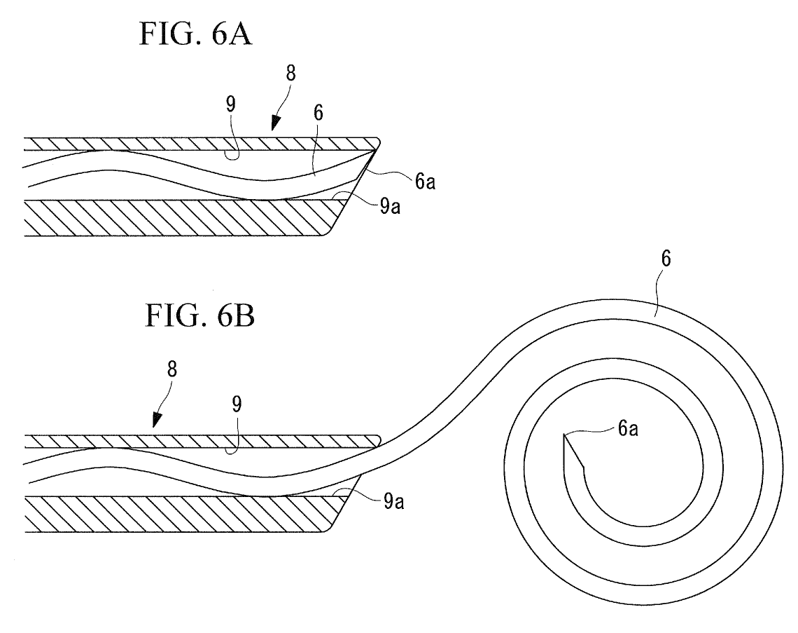

FIG. 6A illustrates a state where the electrode portion 6 is extended into an approximate linear shape and housed in a channel 9 of an insertion portion 8 of the endoscope apparatus 3, while FIG. 6B illustrates a state where the electrode portion 6 is projected from the distal opening 9a of the channel 9 and returns to the original coiled shape of the free state.

On the distal end of the bare wire constituting the electrode portion 6 is provided a pointed sharp portion 6a so that the pericardium A can be readily pierced.

The stopper 7 is a member which is fixed to a certain position of the lead wire 5 and projected radially outward from the lead wire 5.

Next is a description of the endoscope apparatus 3 for setting the thus configured defibrillation electrode 1 according to this embodiment.

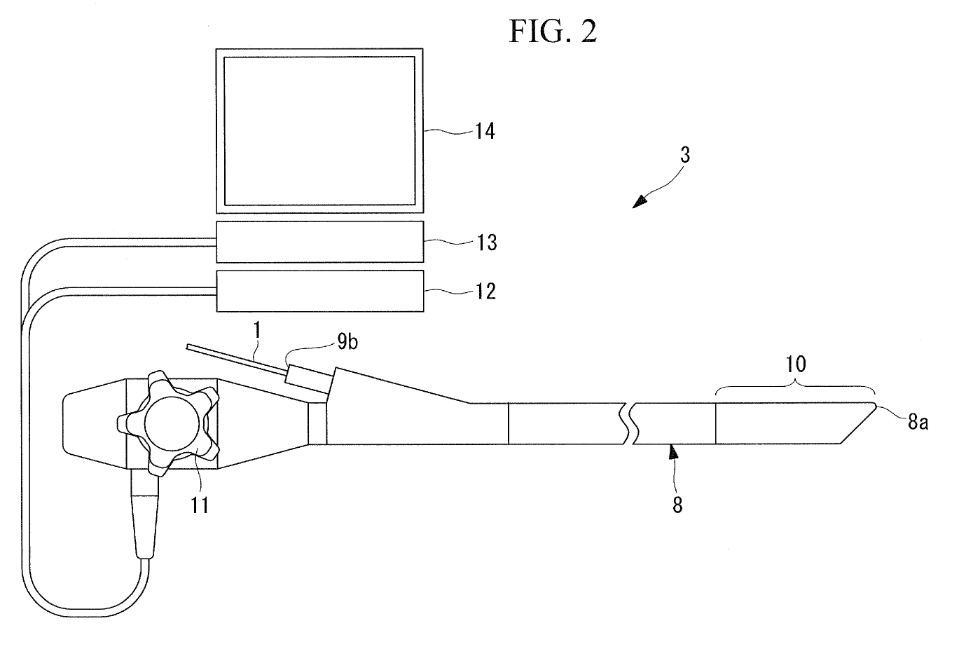

FIG. 2 is an overall configuration diagram of the endoscope apparatus 3. The endoscope apparatus 3 comprises a long and slender insertion portion 8 to be inserted into the body of a patient B, a handle 11 for curving a bendable portion 10 which is provided on the distal end of the insertion portion 8, a light source apparatus 12 for generating illumination light to be guided through the insertion portion 8 and to be emitted from the distal end surface 8a of the insertion portion 8, an image pickup apparatus 13 comprising an imager (not shown) for capturing an image of returning light from the interior of the body of the patient B, and a monitor 14 for displaying the captured image.

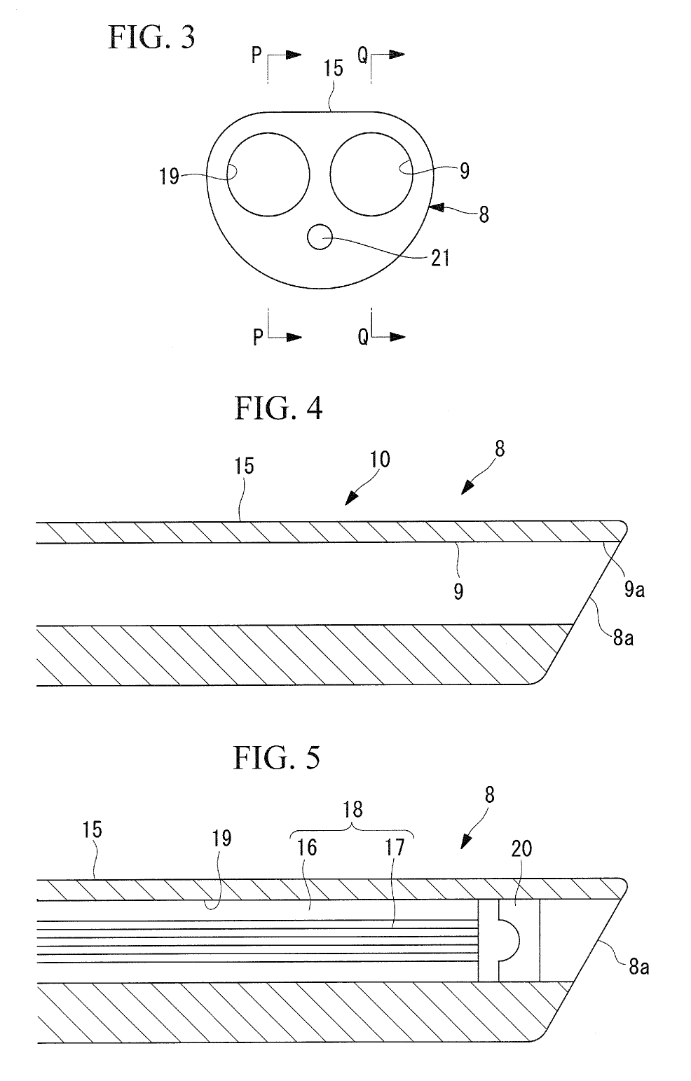

As shown in FIG. 3, the insertion portion 8 has a cross sectional shape the circumferential direction of which partially consists of an approximate linear portion 15. The insertion portion 8 comprises therein: as shown in FIG. 5, a channel 19 for housing a fiber bundle 18 which bundles an illumination fiber bundle 16 for guiding illumination light from the light source apparatus 12 and an image guided fiber bundle 17 for guiding returning light from a living body; and as shown in FIG. 4, the channel 9 for guiding the defibrillation electrode 1.

As shown in FIG. 5, forward of the fiber bundle 18 is provided an object lens 20 for scattering illumination light that has been guided from the light source apparatus 12 to irradiate a living body, and for converging returning light to make it incident into the image guided fiber bundle 17. In FIG. 3, the reference sign 21 denotes a bendable wire for curving the bendable portion 10 as shown in FIG. 6 by applying a pulling force through the operation of the handle 11.

The distal end surface 8a of the insertion portion 8 is unidirectionally inclined. The inclination direction of the distal end surface 8a is opposite to the direction where the abovementioned approximate linear portion 15 is provided in the cross section of the insertion portion 8, across the axis. By so doing, even if the angle of curvature of the bendable portion 10 is small, the distal end surface 8a of the insertion portion 8 can be readily set to face sideways.

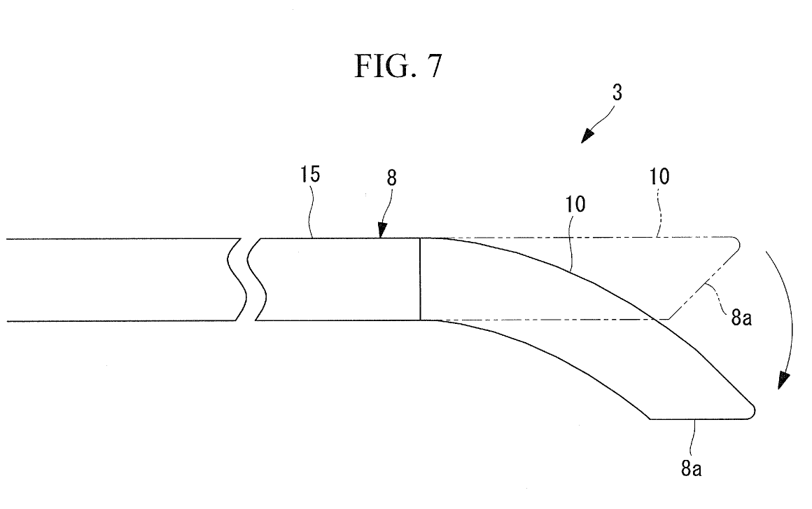

In addition, as shown in FIG. 7, the bendable portion 10 provided on the insertion portion 8 can be curved so that the approximate linear portion 15 can face outward of the curvature. By so doing, when the bendable portion 10 is curved in a state where an outwardly convex object such as the heart C is located on the side of the lateral face on which the approximate linear portion 15 is formed, the lateral face formed with the approximate linear portion 15 can be curved to face outward.

As a result, the contact area between the insertion portion 8 and the surface of the heart C can be kept large as compared to a case where a bendable portion 10 having a circular cross section is curved. Accordingly, an inconvenient rotation of the insertion portion 8 about its axis can be prevented during the curving operation of the bendable portion 10, by which the distal end surface 8a of the insertion portion 8 can be stably faced to a desired direction.

On the lateral side of the handle 11 is provided an insertion port 9b for inserting the defibrillation electrode 1 into the channel 9. The defibrillation electrode 1 can be moved backward and forward through the distal opening 9a of the insertion portion 9 by manually pushing or pulling the defibrillation electrode 1 which extends outward from the insertion port 9b.

Next is a description of the method of cardiac surgery for setting the defibrillation electrode 1 according to this embodiment in the heart C.

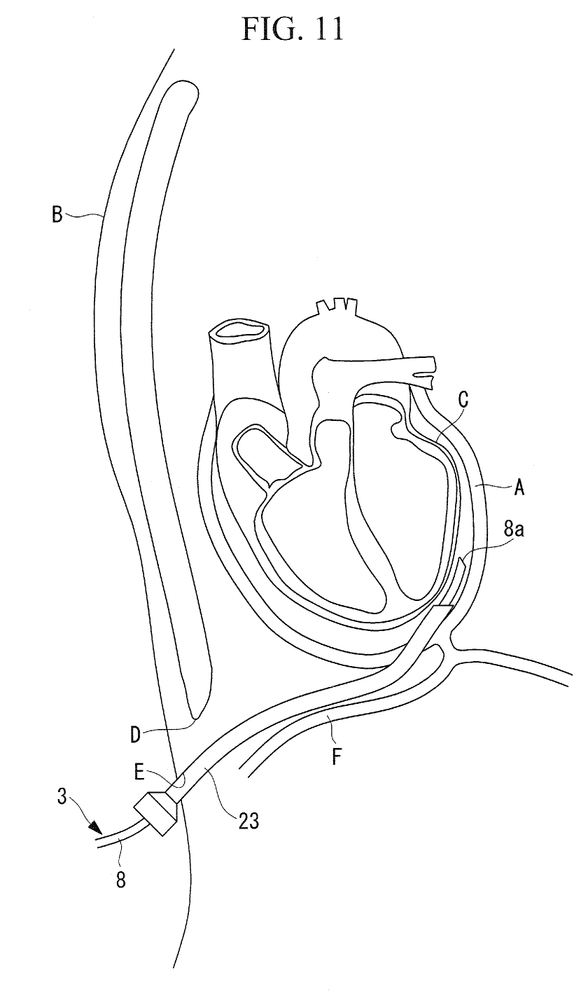

In order to set the defibrillation electrode 1 according to this embodiment in the body of the patient B, firstly as shown in FIG. 9, a step of making a through hole E piercing through the skin below the xiphoid process D in the abdomen of the patient B, and inserting the guide wire 22 therein, is performed. The guide wire 22 is inserted by checking the position of the guide wire 22 in the body of the patient B with an X-ray image pickup apparatus (not shown).

In the example shown in FIG. 9, the guide wire 22 is inserted from the abdomen of the patient B through the space between the diaphragm F and the pericardium A, and then pierces through the pericardium A at the bottom of the pericardium A. The guide wire 22 is inserted until its distal end is located in a space between the pericardium A and the heart C.

In this state, as shown in FIG. 10, a step of inserting a sheath 23 from the through hole E made in the abdomen along the guide wire 22, is performed. Next, in a state where the sheath 23 has been inserted so that its distal end can be located in a space between the pericardium A and the heart C, a step of withdrawing the guide wire 22 is performed.

Next, as shown in FIG. 11, a step of inserting the insertion portion 8 of the endoscope apparatus 3 into the sheath 23, is performed. The distal end of the insertion portion 8 is moved forward within the pericardium A while displaying on the monitor 14 an image that has been acquired by capturing returning light resulting from irradiation on the interior of the body of the patient B with illumination light generated from the light source apparatus 12, using the endoscope apparatus 3 the insertion portion 8 of which has been inserted. At this time, the insertion portion 8 is moved so that the approximate linear portion 15 provided on the insertion portion 8 can face the heart C side.

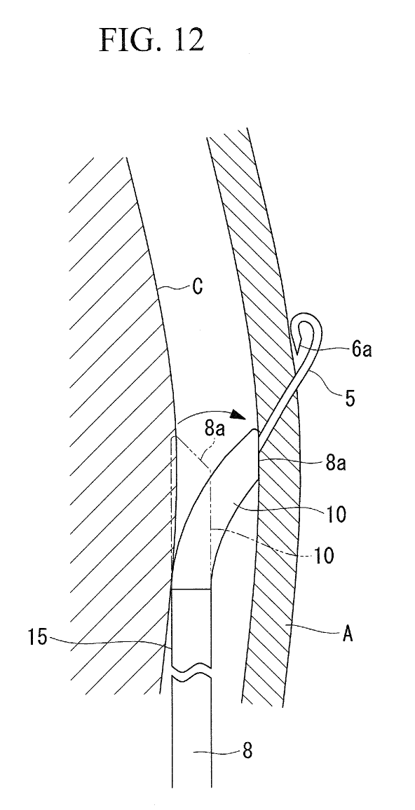

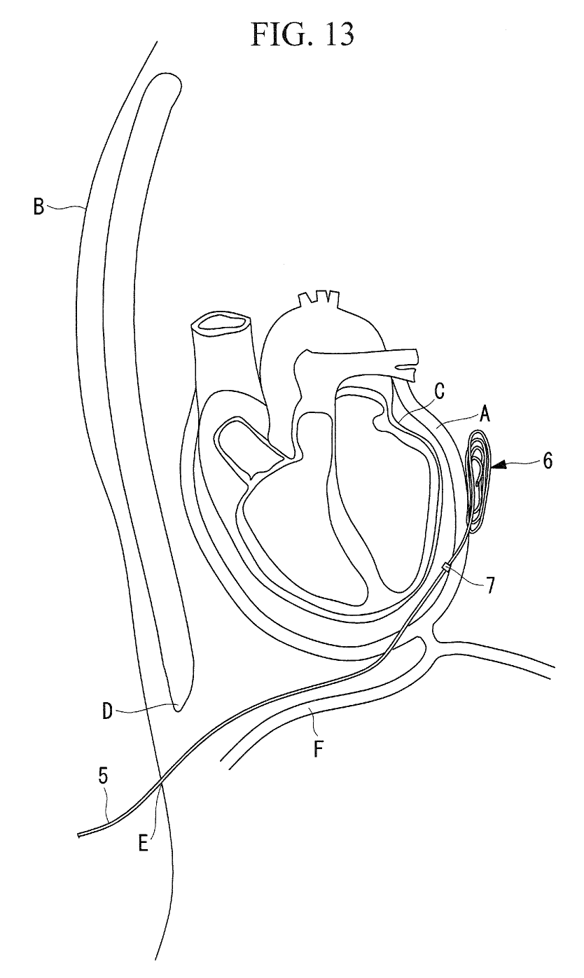

Then, in a state where it has been confirmed that the distal end of the insertion portion 8 is placed at a desired position on the left ventricle of the heart C by using the monitor 14, as shown in FIG. 12 a step of curving the bendable portion 10 is performed through the operation of the handle 11. Since the distal end surface 8a of the insertion portion 8 is inclined oppositely to the approximate linear portion 15 across the axis, the distal end surface 8a of the insertion portion 8 can be readily confronted with and attached to the inner surface of the pericardium A by curving the bendable portion 10 only at a relatively small angle so that the approximate linear portion 15 can face outward.

In this state, a step of manually pushing the defibrillation electrode 1 extending outward from the insertion port 9b that is located in a vicinity of the handle 11 of the endoscope apparatus 3 so as to project the distal end of the defibrillation electrode 1 from the distal end of the insertion portion 8, is performed. Since the distal end of the defibrillation electrode 1 is provided with the sharp portion 6a, the defibrillation electrode 1 projecting from the distal opening 9a of the channel 9 on the distal end surface 8a pierces through the pericardium A with its sharp portion 6a, and its distal end is placed outside the pericardium A.

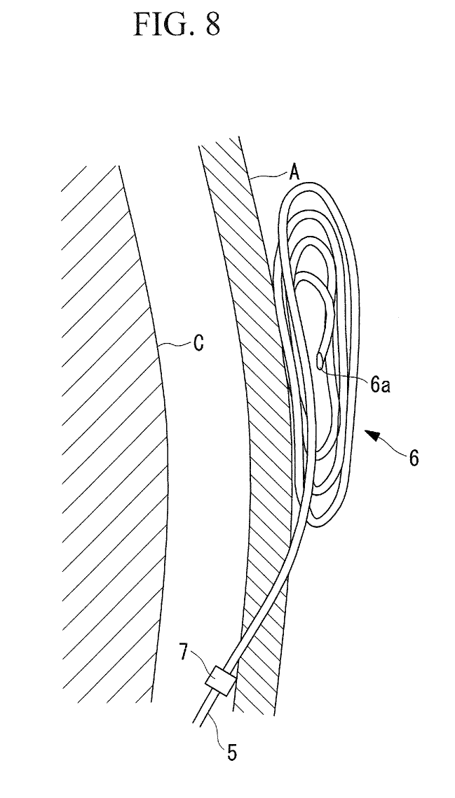

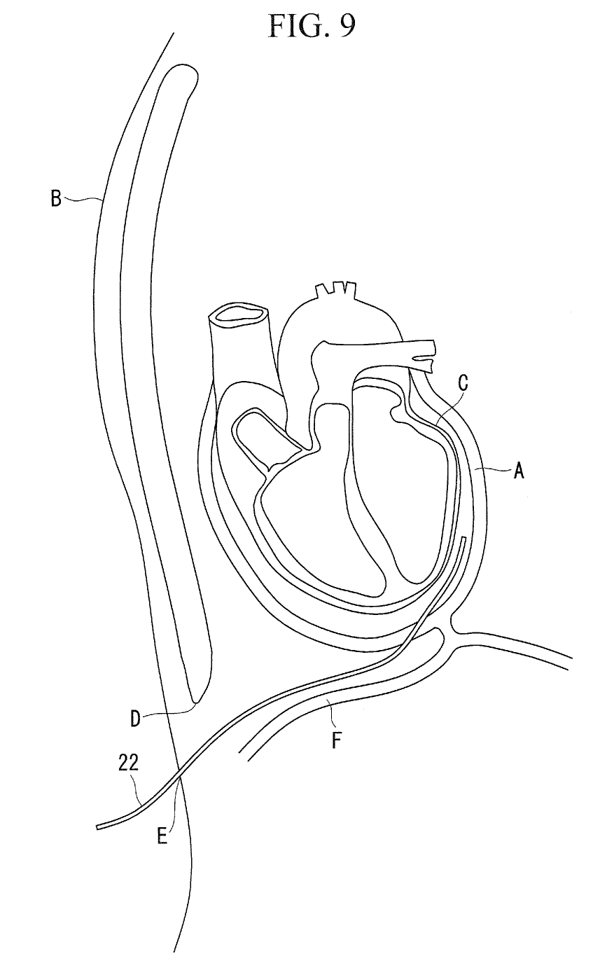

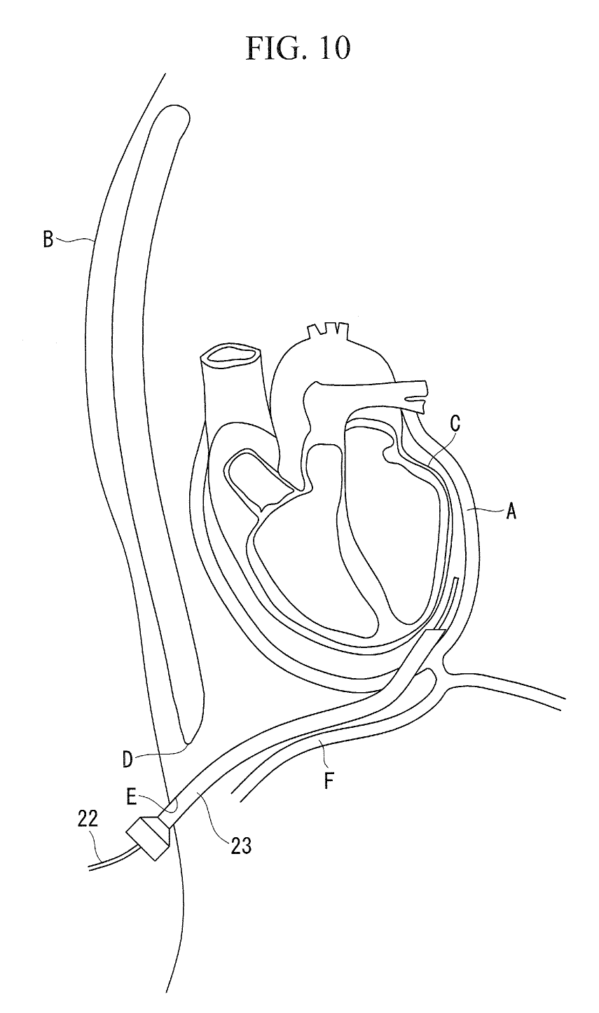

Then, by further performing the step of pushing the defibrillation electrode 1, as shown in FIG. 8, the whole of the coiled electrode portion 6 is sent out to the outside of the pericardium A. Since the stopper 7 is provided in a vicinity of the electrode portion 6, the stopper 7 abuts against the pierced part in the pericardium A to thereby hinder any further movement of the defibrillation electrode 1.

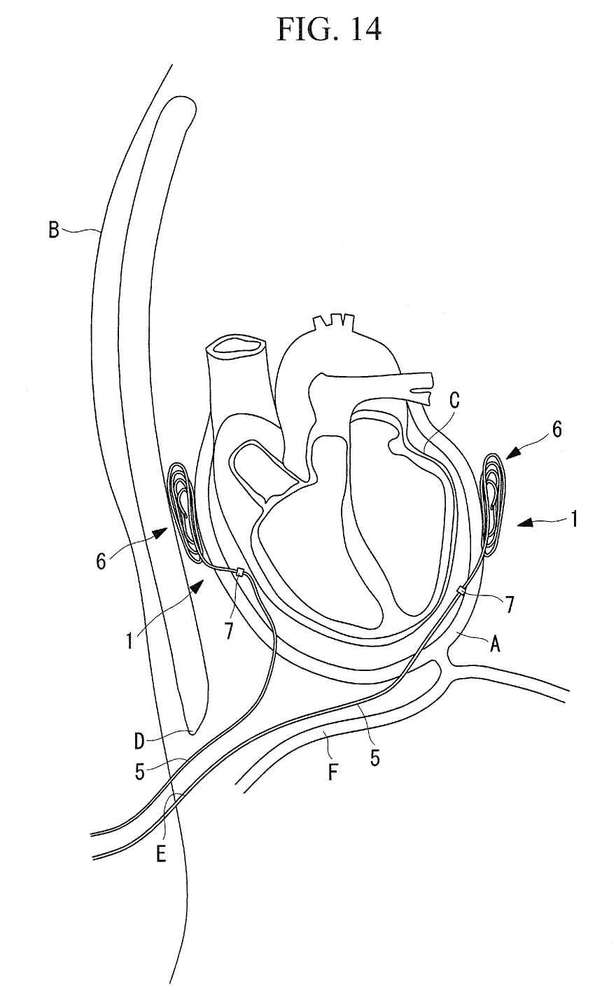

After this step, a step of withdrawing the insertion portion 8 is performed, and then a step of withdrawing the sheath 23 is performed, by which, as shown in FIG. 13, the defibrillation electrode 1 can be set in a state where the electrode portion 6 is placed outside the pericardium A facing the left ventricle.

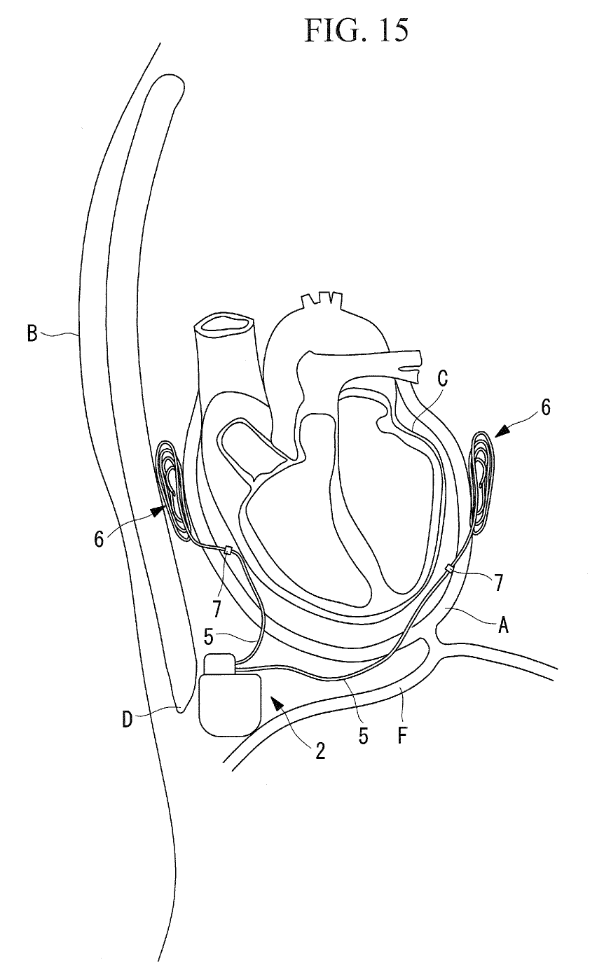

In addition, upon the placement of the electrode portion 6 at a position facing the right ventricle, the defibrillation electrode 1 can also be readily and reliably set as shown in FIG. 14, similarly to the abovementioned manner, by performing the respective steps of placing the guide wire 22, inserting the sheath 23, inserting the insertion portion 8 of the endoscope apparatus 3 through the interior of the sheath 23, inserting the defibrillation electrode 1 through the endoscope apparatus 3, piercing through the pericardium A with the defibrillation electrode 1, placing the electrode portion 6 which is spread in a coiled shape outside the pericardium A, interposing the pericardium A between the electrode portion 6 and the stopper 7, and withdrawing the insertion portion 8 and the sheath 23. Then, by connecting the two lines of lead wires 5 that have been fixed through the through hole E made in the abdomen to the defibrillator mainbody 4, and by implanting the defibrillator mainbody 4 inside the xiphoid process D of the patient B through the same through hole E as shown in FIG. 15, the body implantable defibrillator 2 can be set.

In this manner, according to the method of cardiac surgery of this embodiment, the guide wire 22, the sheath 23, and the insertion portion 8 are introduced to desired positions on the pericardium A through a space between the pericardium A and the heart C. By so doing, there is no need of complicated operations for separating the tightly contacted or adhered tissues in the thoracic cavity and moving devices barely through narrow spaces therebetween, and the devices can readily reach desired positions on the pericardium A. In addition, since there is no need of newly retaining a space in the thoracic cavity through single lung ventilation, physical burdens on the patient B can be alleviated.

Moreover, according to the defibrillation electrode 1 of this embodiment, the lead wire 5 pierces through the pericardium A, and thereby the pericardium A is interposed between the electrode portion 6 that is spread in a coiled shape outside the pierced part and the stopper 7 placed inside the pierced part. Therefore, the position of the electrode portion 6 can be fixed without moving regardless of the heartbeat of the heart C. As a result, fibrillation of the heart C can be effectively removed by applying a defibrillation voltage to a desired site of the heart C. In particular, since the relatively largely spread electrode portions 6 are placed outside the pericardium A, inconvenient interference with the heartbeat of the heart C due to the electrode portions 6 can be prevented.

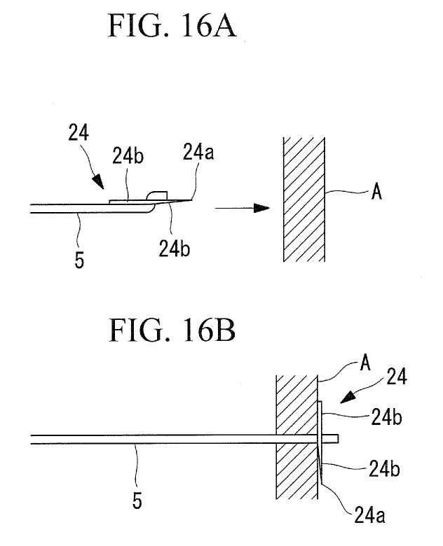

In the defibrillation electrode 1 according to this embodiment, the distal end of the lead wire 5 constituting the defibrillation electrode 1 is used as the pointed sharp portion 6a so that the distal end of the lead wire 5 can pierce through the pericardium A. However, instead of this, as shown in FIG. 16A and FIG. 16B, the distal end of the lead wire 5 may also be provided with a stopper 24 having a pointed sharp portion 24a and a projected portion 24b.

This stopper 24 is a plate-like member attached to the distal end of the lead wire 5. Before piercing through the pericardium A, for example, the stopper 24 is placed inside the channel 9 or an armored tube 25 that will be described later, and as shown in FIG. 16A the stopper 24 is placed along the lead wire 5 so that the sharp portion 24a faces forward and the projected portion 24b is not projected in a radial direction of the lead wire 5. By so doing, the pericardium A can be readily pierced by pushing the lead wire 5 in the longitudinal direction.

On the other hand, after piercing through the pericardium A, as shown in FIG. 16B this stopper 24 is placed orthogonally to the lead wire 5 and the projected portion 24b is projected radially outward to limit the backward movement of the lead wire 5.

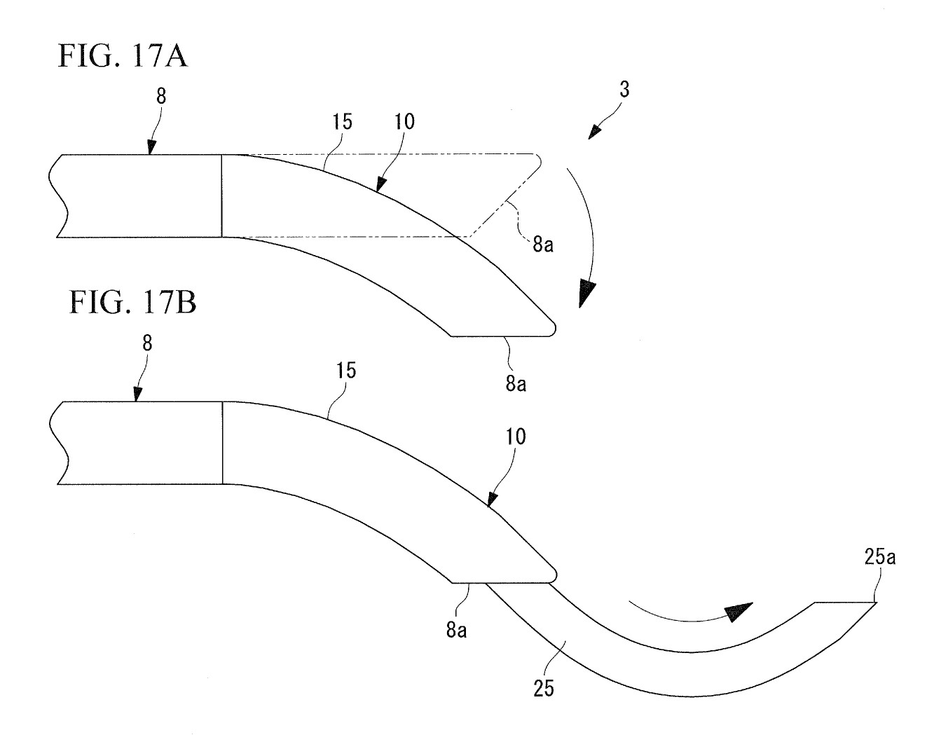

In order to attach the thus configured defibrillation electrode 1, the endoscope apparatus 3 as shown in FIG. 17A and FIG. 17B are used. That is to say, as shown in FIG. 17A, this endoscope apparatus 3 comprises the armored tube 25 which is movable in the channel 9 inside the bendable portion 10 which can be unidirectionally curved similarly to FIG. 7, and inside the armored tube 25 is housed the lead wire 5 in an extended state. As shown in FIG. 17B, a pointed sharp portion 25a is provided on the distal end of the armored tube 25, and the armored tube 25 is finished to be curved oppositely to the direction of curvature of the bendable portion 10 of the insertion portion 8 of the endoscope apparatus 3 when projected from the distal opening 9a of the channel 9.

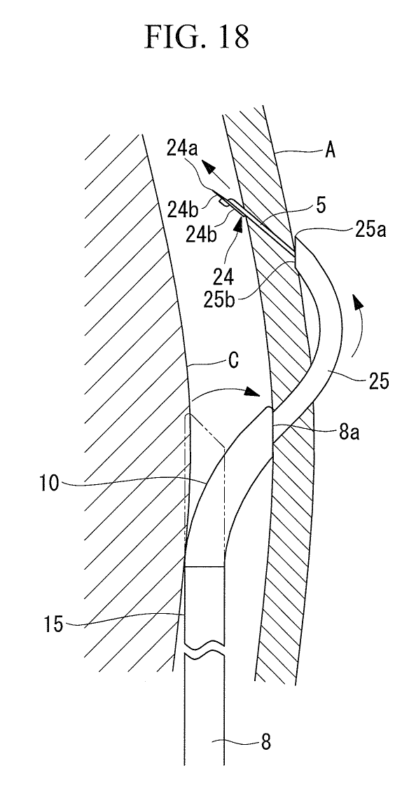

By so doing, as shown in FIG. 18, by projecting the armored tube 25 from the inside of the channel 9 in a state where the bendable portion 10 is curved so that the distal end surface 8a can tightly contact to the pericardium A, the sharp portion 25a of the armored tube 25 pierces through the pericardium A to project outside the pericardium A, and then is curved oppositely to the direction of curvature of the bendable portion 10. Therefore, the distal end surface 25b is tightly contacted to the outer surface of the pericardium A. In this state, by projecting the lead wire 5 from the distal end surface 25b of the armored tube 25, the sharp portion 24a of the stopper 24 attached to the distal end of the lead wire 5 pierces through the pericardium A and enters inside the pericardium A.

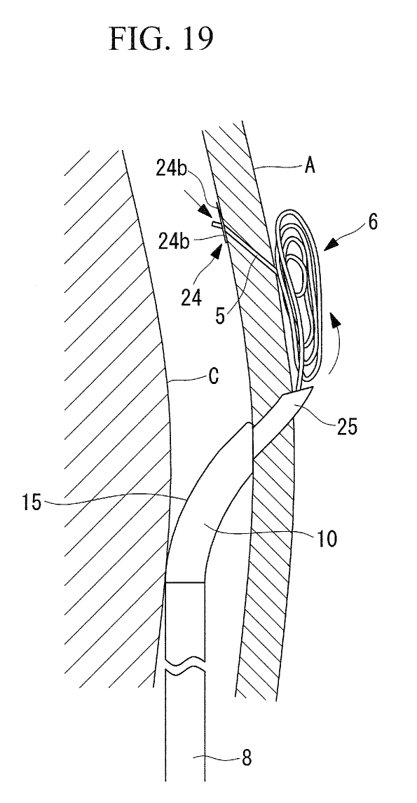

Then, in a state where the stopper 24 has completely entered inside the pericardium A, the lead wire 5 is moved slightly backward as shown in FIG. 19. By so doing, the stopper 24 is opened orthogonally to the lead wire 5 and abuts against the inner surface of the pericardium A to thereby limit the backward movement of the lead wire 5.

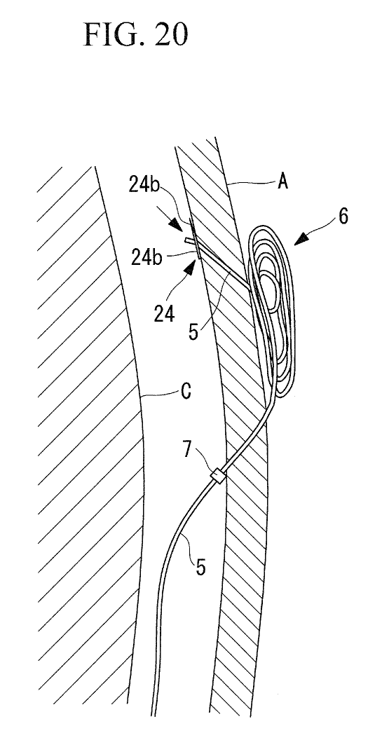

Thereafter, the coiled electrode portion 6 is released outside the pericardium A by sending out the lead wire 5 from the inside of the armored tube 25 as well as pulling back the armored tube 25 into the channel 9 of the insertion portion 8. Then, by withdrawing the insertion portion 8 after the armored tube 25 has been completely housed in the channel 9, as shown in FIG. 20 the electrode portion 6 is placed outside the pericardium A, and is reliably fixed without moving by the stoppers 7 and 24 provided on both sides thereof.

In addition, in the abovementioned embodiment, the interior of the armored tube 25 and the interior of the channel 9 of the insertion portion 8 whose distal end surface 8a is to be tightly contacted to the pericardium A may be sucked to produce a decompressed state. By so doing, the operation of piercing the pericardium A with the defibrillation electrode 1 can be facilitated in a state where the distal end surface 8a or 25b of the insertion portion 8 or the armored tube 25 is tightly contacted to the surface of the pericardium A.

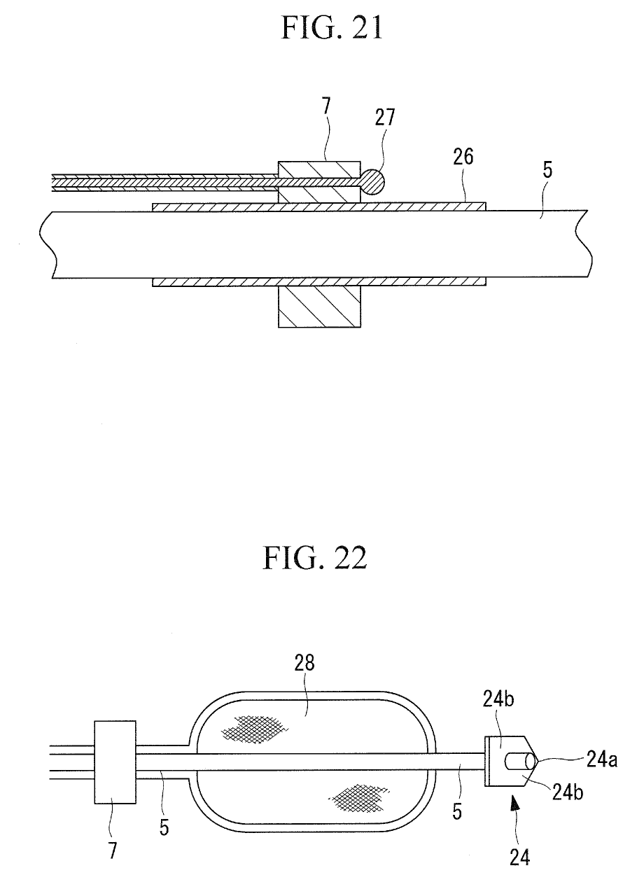

Moreover, in this embodiment, as shown in FIG. 21, the stopper 7 may be insulated from the lead wire 5 by using an insulation material 26, and a pacing electrode portion 27 may be fixed to the stopper 7. By so doing, the pacing electrode portion 27 can be fixed to the pericardium A separately from the electrode portion 6 of the defibrillation electrode 1, and therefore the electrocardiographic signal can be more accurately detected to output an appropriate pacing signal.

Furthermore, in this embodiment, the coil-shaped electrode portion 6 is exemplified. However, instead of this, as shown in FIG. 22, a mesh-shaped electrode portion 28 made of a conductive metal material which is planarly spread when released from the inside of the channel 9 or the inside of the armored tube 25, may also be employed.

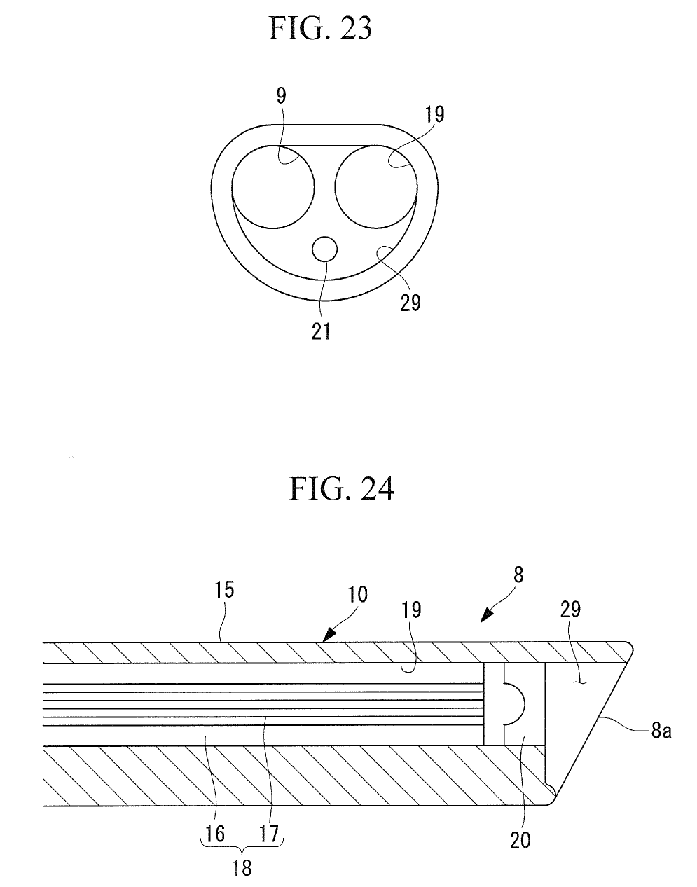

In this embodiment, the channel 19 for housing the observation optical system and the channel 9 for backward and forward movement of the defibrillation electrode 1 are independently provided along the way to the distal end surface 8a of the insertion portion 8 of the endoscope apparatus 3. However, instead of this, as shown in FIG. 23 and FIG. 24, a space 29 for merging these two channels 9 and 19 may be provided in the distal portion of the insertion portion 8. By so doing, even in a state where the distal end surface 8a of the insertion portion 8 is tightly contacted to the pericardium A, the manners of the defibrillation electrode 1 and the armored tube 25 when being sent out from the inside of the channel 9 and when piercing through the pericardium A, can be observed.

In addition, the object lens 20 may also be arranged in the inclined distal end surface 8a of the insertion portion 8. By so doing, the visual field of the observation optical system can be maximized.

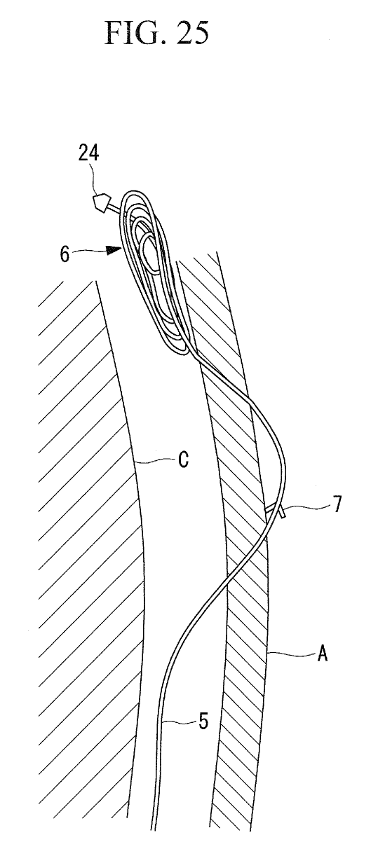

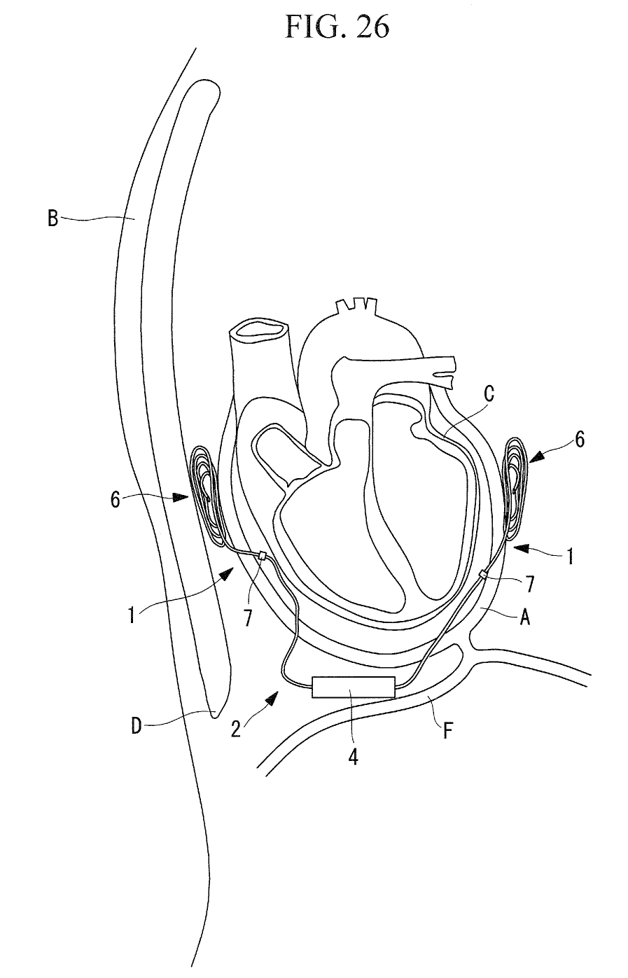

Moreover, in this embodiment, the electrode portion 6 spread in a coiled shape is placed outside the pericardium A. However, instead of this, as shown in FIG. 25, the electrode portion 6 may also be placed inside the pericardium A by piercing through the pericardium A twice with the lead wire 5 like a sewing procedure. In this case, the stopper 7 is preferably made of an elastic member which projects radially outward after piercing through the pericardium A.

Furthermore, as shown in FIG. 26, by employing a thin and compact defibrillator mainbody 4 of the defibrillator 2, the defibrillator mainbody 4 can be set in a gap between the pericardium A and the diaphragm F.

In this embodiment, upon placement of the electrode portion 6 outside the pericardium A, the lead wire 5 is sent out in a state where the distal end of the insertion portion 8 is placed inside the pericardium A. However, instead of this, the lead wire 5 may also be sent out in a state where the distal end of the insertion portion 8 is placed outside the pericardium A.

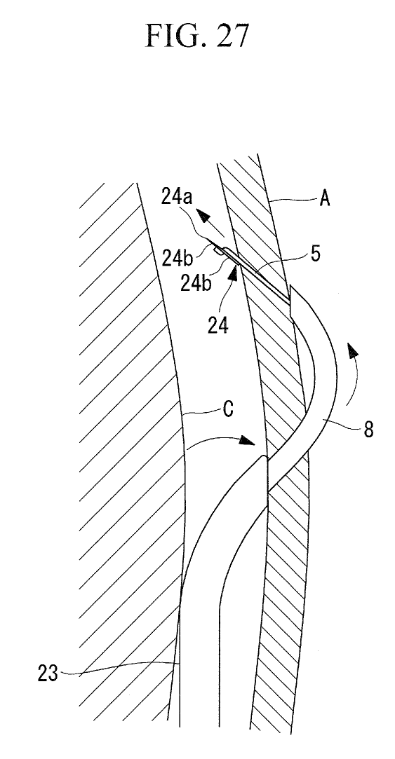

FIG. 27 illustrates an example of a method for setting the electrode portion 6 in a state where the distal end of the insertion portion 8 is placed outside the pericardium A. By so doing, upon setting of the electrode portion 6 on the pericardium A, the operation can be done by checking the position and the state of the electrode portion 6 in an endoscopic image.

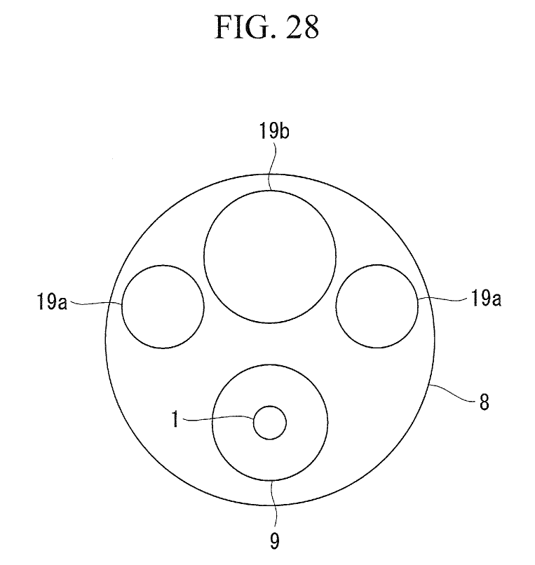

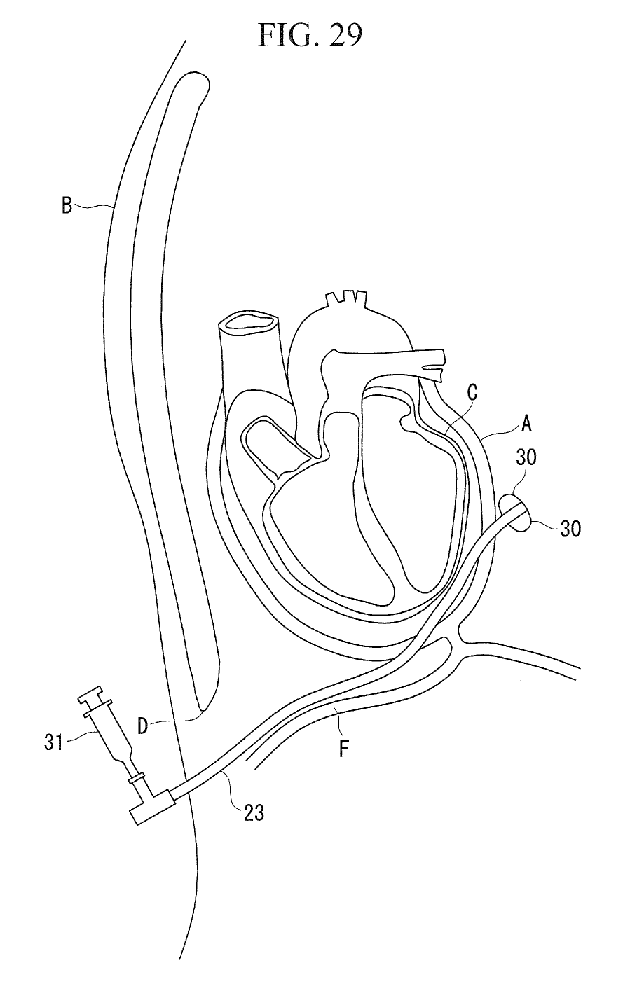

The insertion portion 8 of the endoscope apparatus 3 used herein may be either the insertion portion 8 according to the abovementioned embodiment, or a conventional type of an insertion portion 8 having a circular cross sectional shape. FIG. 28 illustrates an example of the insertion portion 8 having such a circular cross sectional shape, which comprises a channel 19a for housing an illumination fiber bundle, a channel 19b for housing an image guided fiber bundle, and a channel 9 for housing the defibrillation electrode 1.

In addition, the method for placing the distal portion of the insertion portion 8 outside the pericardium A can be performed by, for example, inserting the sheath 23 up to a vicinity of the setting position of the electrode portion 6, then pushing out the defibrillation electrode 1 from the inside of the channel 9 to pierce through the pericardium A, and in this state, pushing out the insertion portion 8 forward. In this case, the sheath 23 preferably has its distal end surface unidirectionally inclined similarly to the insertion portion 8 of the endoscope apparatus 3 according to this embodiment, and is finished to be curved so that the distal end surface can face the inner circumference side. By so doing, when the distal end surface of the sheath 23 is tightly contacted to the inner surface of the pericardium A, the defibrillation electrode 1 and the insertion portion 8 can be pushed out toward the pericardium A. Therefore, these operations can be facilitated.

Moreover, in this embodiment, the sheath 23 may also be placed outside the pericardium A by piercing through the pericardium A with the distal end of the sheath 23 that has been inserted into the pericardium A.

FIG. 29 illustrates an example where the distal portion of the sheath 23 that has been inserted from the bottom of the xiphoid process D into the pericardium A is placed on the left ventricle side outside the pericardium A. By so doing, when insertion and withdrawal of the sheath 23, the endoscope apparatus 3, and the like are repeated in the pericardium A, the manipulations of these devices can be facilitated and influences to the heart C caused by these devices scratching the heart C can be prevented.

In this case, a balloon 30 is provided on the distal portion of the sheath 23, and the position of the distal portion of the sheath 23 relative to the pericardium A can be fixed by expanding the balloon 30 after the distal portion of the sheath 23 is placed outside the pericardium A. As to the method for expanding and deflating the balloon 30, for example, there is employed a method of injecting a liquid into or discharging it from a water pipe conduit which is formed inside the lateral wall of the sheath 23 and is communicated to the interior of the balloon 30, by using a syringe 31 or the like. In addition, the sheath 23 preferably has an adjustable flexibility so that the sheath 23 can be flexibly curved to fit with the shapes of the heart C and the pericardium A when being inserted and withdrawn and a desired shape can be kept when the defibrillation electrode 1 is being set. For example, the flexibility of the sheath 23 can be readily adjusted inside the pericardium A by providing a coiled spring which is spiral along the inner circumferential face likewise of a conventional stylet, and changing the compression of the coiled spring.

Furthermore, in this embodiment, a part of a lung may be compressively deformed in a vicinity of the operation site by using a catheter 32 which comprises a compressive deformation device on its distal end, or the like. As for the compressive deformation device, a conventional device such as the balloon 30 is employed.

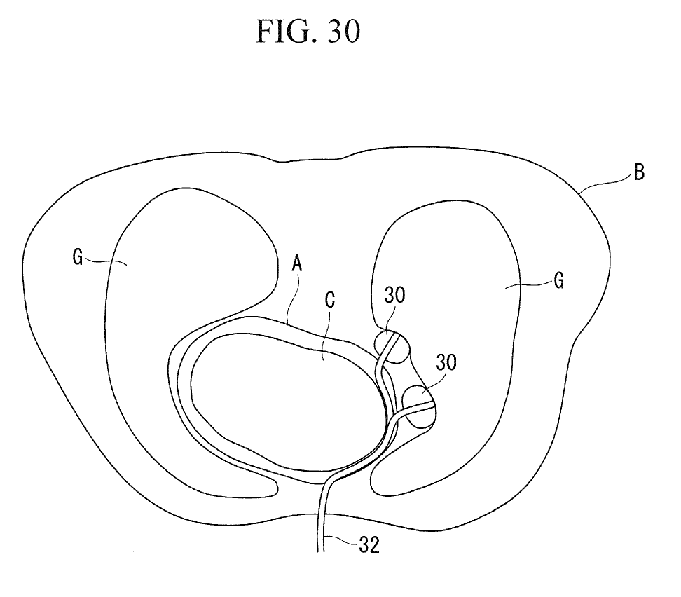

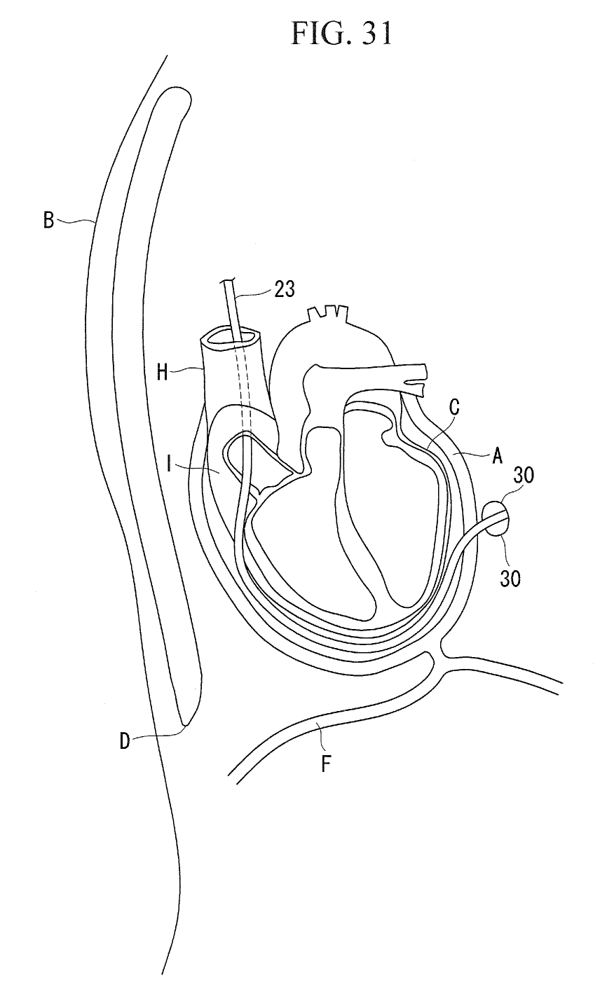

FIG. 30 illustrates an example where two catheters 32 are inserted from the bottom of the xiphoid process D into the pericardium A, the balloons 30 are placed to interpose the operation site outside the pericardium A, and these balloons are expanded. By so doing, a part of a lung G in a vicinity of the pericardium A can be compressively deformed, and thereby a space can be readily retained in the operation site without halting the ventilation of the lungs G.

The method for setting the defibrillation electrode 1 on the pericardium A is not limited to the abovementioned embodiment in which the insertion is carried out from the bottom of the xiphoid process D into the pericardium A, and the insertion into the pericardium A may also be carried out in an transvenous manner.

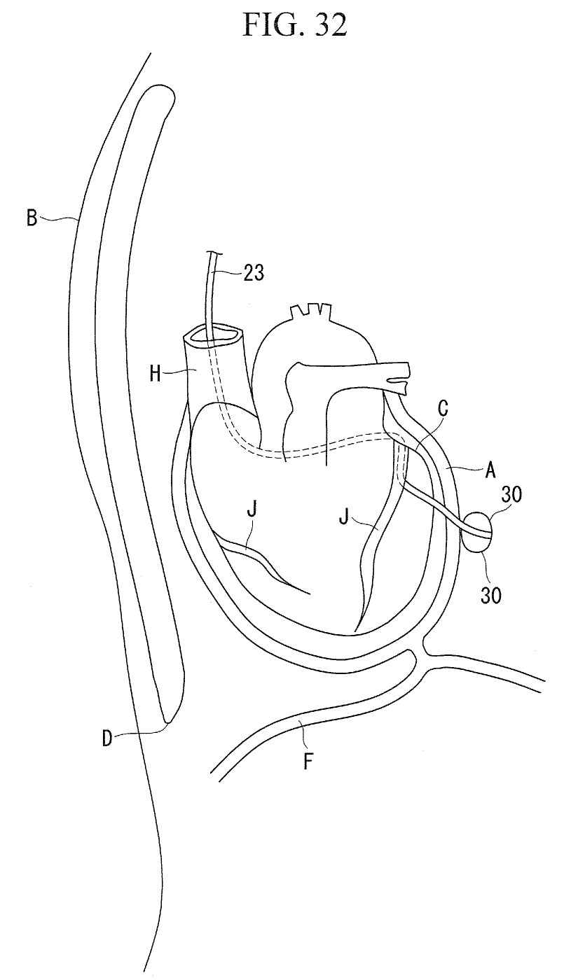

FIG. 31 illustrates a method in which the sheath 23 is transvenously inserted from the interior of the superior vena cava H into the right atrium, piercing through the cardiac wall in the right auricle I, and inserted into the pericardium A, and then its distal end is placed outside the pericardium A. This method is also capable of setting the defibrillation electrode 1 outside the pericardium A in a low invasive manner to the body of the patient B.

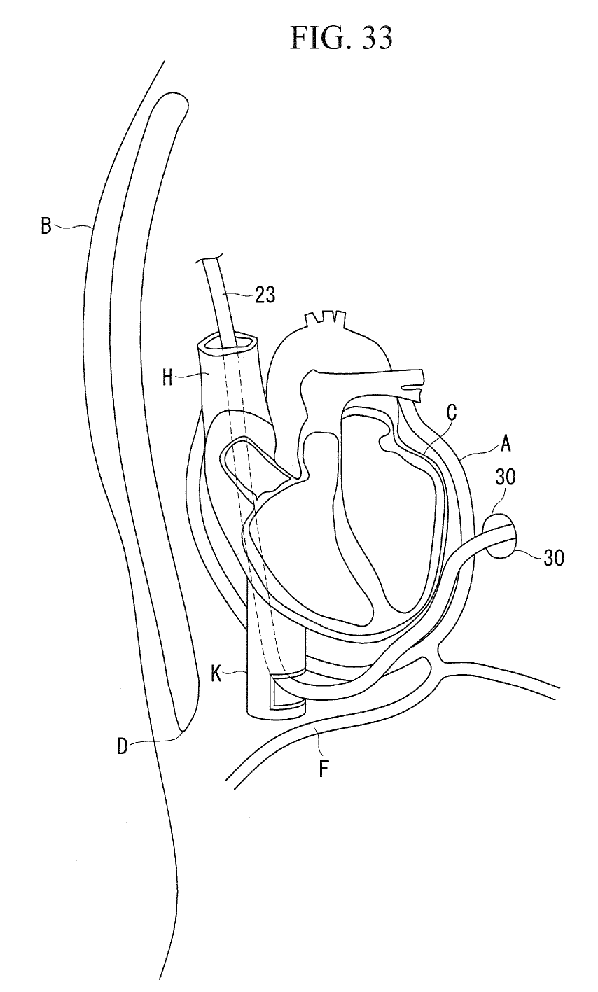

FIG. 32 illustrates a method in which the sheath 23 is transvenously inserted from the interior of the superior vena cava H into a coronary vein J through the right atrium, and then inserted into the pericardium A by piercing through the vascular wall of the coronary vein J with its distal end. In the case where the sheath 23 is inserted from the right auricle I into the pericardium A, the insertion position into the pericardium A is limited. However, the insertion position into the pericardium A can be selected by inserting the sheath 23 from either one of coronary veins J located on the cardiac wall into the pericardium A. In addition, since the sheath 23 is guided along the coronary vein J, the sheath 23 can be readily inserted to the target insertion position.

FIG. 33 illustrates a method in which the sheath 23 is transvenously inserted from the superior vena cava H into the inferior vena cava K through the right atrium, and piercing through the vascular wall of the inferior vena cava K outside the pericardium A, and then inserted through the pericardium A to the interior of the pericardium A. In this case, since no vascular wall is pierced within the pericardium A, bleeding in the pericardium A can be prevented.

As mentioned above, the method of cardiac surgery according to the present invention has been described by exemplifying methods for setting the defibrillation electrode 1 in the heart C. However, the method of cardiac surgery of the present invention may also be applied to other cardiac surgeries.

Another example of the method of cardiac surgery of the present invention is a coronary artery bypass surgery which uses a part of an internal thoracic artery as a graft.

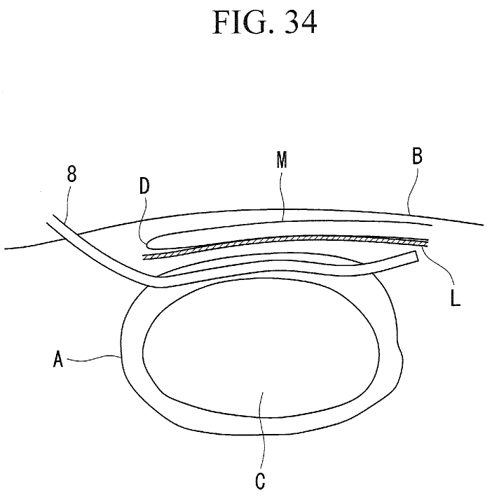

FIG. 34 illustrates an example in which the coronary artery bypass surgery is carried out by using the method of cardiac surgery of the present invention. After the insertion portion 8 of the endoscope apparatus 3 has been inserted from the bottom of the xiphoid process D into the pericardium A, the pericardium A is pierced in a vicinity of an internal thoracic artery L to be resected, and thereby the insertion portion 8 is placed outside the pericardium A. Then, a part of the internal thoracic artery L is resected by introducing an appropriate treatment tool to the position of the internal thoracic artery L through the channel inside the insertion portion 8, and the resected internal thoracic artery L is separated from the breast bone M and brought into the pericardium A. The thus collected part of the internal thoracic artery L is connected to a coronary artery within the pericardium A by using a similar surgery method to that of conventional methods. By so doing, the coronary artery bypass surgery can be carried out without performing open-chest approaches simply in a low invasive manner.

The present invention has the following aspects.

A first aspect of the present invention provides a method of cardiac surgery, comprising: using a device which comprises a treatment unit to be inserted into a thoracic cavity for performing treatment of a heart; inserting a distal end of this device between the heart and a pericardium; thereafter piercing through the pericardium with the distal end of the device; and performing treatment of the heart from the outside of the pericardium by using the treatment unit.

According to the first aspect of the present invention, the treatment of the heart can be performed from the outside of the pericardium by inserting the device between the heart and the pericardium, and then piercing through the pericardium with its distal end to thereby place the treatment unit outside the pericardium. In this case, the device is moved along a space between the heart and the pericardium up to a desired position on the pericardium. By so doing, interference with the manipulation of the device due to surrounding tissues around the heart can be avoided, and thereby the surgery can be facilitated. Also, the necessities of open-chest approaches and single lung ventilation can be avoided, and thereby physical burdens on the patient can be alleviated.

In the abovementioned aspect, the device may also be inserted between the heart and the pericardium by inserting the device from the bottom of a xiphoid process into the thoracic cavity and piercing through the pericardium with the distal end of the device.

The device can be readily introduced to the pericardium in a low invasive manner by inserting the device from the bottom of the xiphoid process near the pericardium.

In addition, in the abovementioned aspect, the device may also be inserted between the heart and the pericardium by inserting the device into a right atrium and piercing through a cardiac wall of a right auricle with the distal end of the device.

By so doing, the device can be readily introduced between the heart and the pericardium in a low invasive manner.

Moreover, in the abovementioned aspect, the device may also be inserted between the heart and the pericardium by inserting the device from the interior of the right atrium into a vein communicated to the interior of a right atrium and located on the cardiac wall, and piercing through the vascular wall of the vein with the distal end of the device.

By so doing, the device can be inserted from a desired position among where veins are located, to a space between the pericardium and the heart.

Furthermore, in the abovementioned aspect, the device may also be inserted between the heart and the pericardium by piercing through a vascular wall of a vein located outside the pericardium from the interior of the vein, and then piercing through the pericardium, with the distal end of the device.

By so doing, since no vascular wall is pierced by the device in the pericardium, bleeding in the pericardium can be prevented.

In addition, in the abovementioned aspect, a cylindrical member into which the device is removably insertable is inserted between said heart and said pericardium, and thereafter said device is inserted between said heart and said pericardium through an interior of the cylindrical member.

By so doing, the pathway of the device can be retained in the pericardium, and even upon repetition of insertion and withdrawal of the device, influences to the heart can be prevented and the manipulation of the device can be facilitated.

Moreover, in the abovementioned aspect, a space for manipulating the treatment unit may be retained outside the pericardium by compressively deforming a lung using a balloon placed between the pericardium and the lung.

By so doing, a necessary space can be retained outside the pericardium without halting the ventilation of lungs.

A second aspect of the present invention provides a method of cardiac surgery, comprising: inserting a lead wire of a defibrillator between the pericardium and the heart; thereafter piercing through the pericardium with the distal end of the lead wire; and placing an electrode provided on the distal end of the lead, outside the pericardium.

According to the second aspect of the present invention, the electrode of the defibrillator can be readily placed outside the pericardium as well as reducing the invasion into the body of the patient. Moreover, displacement of the electrode due to the movement of the heart which is beating within the pericardium can be prevented by placing the electrode outside the pericardium.

A third aspect of the present invention provides a method of cardiac surgery, comprising: inserting a device for bypassing a coronary artery with use of an internal thoracic artery as a graft, between the heart and the pericardium; thereafter piercing through the pericardium with the distal end of the device to thereby introduce the device to the internal thoracic artery; separating the internal thoracic artery; and connecting the ends of the internal thoracic artery to the coronary artery.

By so doing, the device can be readily introduced to the position of the internal thoracic artery without performing open-chest approaches, and the coronary artery bypass surgery can be performed in a low invasive manner.

A fourth aspect of the present invention provides a defibrillation electrode, comprising: an electrode portion to be placed along a surface of a pericardium; a lead wire which is connected to the electrode portion for piercing through the pericardium; and a stopper which is provided on the lead wire to be placed in a vicinity of the pierced part of the pericardium in a state where the electrode portion is placed on the surface of the pericardium, and then projected radially outward from the lead wire to limit a longitudinal movement of the lead wire.

According to the fourth aspect of the present invention, when the electrode portion is placed on the surface of the pericardium in a state where the lead wire pierces through the pericardium, the radially outwardly projecting stopper abuts against the pierced part of the pericardium to thereby limit the longitudinal movement of the lead wire. Therefore, the electrode portion is kept in a fixed state to the pericardium. Accordingly, regardless of the movement of the heart relative to the pericardium, the position of the electrode portion can be kept in a fixed position, by which the defibrillation voltage can be effectively applied to the heart.

In the abovementioned aspect, the electrode portion may also comprise an elastic member the external dimension of which is expanded or contracted by its elasticity.

By so doing, the elastic member can be housed in a contracted state within a channel or a sheath of an endoscope having a long and slender insertion portion, and the electrode portion can be placed in a relatively widely contacted state with the pericardium by inserting the insertion portion in a vicinity of the pericardium, and then pushing out the elastic member from the interior of the channel or the sheath to thereby expand the elastic member. By so doing, the setting operation can be facilitated, and the defibrillation voltage can be effectively applied to the heart.

In addition, in the abovementioned aspect, the elastic member may also consist of a part of the lead wire which is designed to take an approximate linear shape by applying an external force and to take a coiled shape by releasing the external force.

By so doing, since the part of the lead wire is formed in a coiled shape in a released state, it can be housed in an approximately linearly extended state within a channel or a sheath of a long and slender insertion portion of an endoscope, only by pushing it out from the interior of the channel or the sheath, and by expanding the part of the lead wire in a coiled shape.

Moreover, in the abovementioned aspect, the stopper may be fixed to the lead wire by having a space between the stopper and the electrode portion to a degree capable of interposing the pericardium.

By so doing, when the lead wire between the electrode portion and the stopper is arranged to pierce through the pericardium, the electrode portion is placed on either the outer surface or the inner surface of the pericardium, and the stopper is placed on the opposite side of the electrode portion across the pericardium. Therefore, the pericardium is interposed between the electrode portion and the stopper, and the electrode portion can be more reliably fixed to the pericardium.

Furthermore, in the abovementioned aspect, the layout may also be such that the lead wire is provided on both sides of the electrode portion, two of the stoppers are provided on the lead wire on both sides of the electrode portion, and the stopper on the distal side comprises a sharp portion which is arranged at the front end to pierce through the pericardium with the lead wire, and a projected portion which is arranged at the rear end to limit the backward insertion through the pericardium.

By so doing, when the pericardium is pierced with the sharp portion of the stopper provided on the distal side twice, the projected portion provided at the rear end of the stopper prevents the lead wire from being withdrawn in the opposite direction, and the electrode portion is fixed to the pericardium. By respectively piercing through the pericardium with the lead wire provided on the both sides of the electrode portion and by limiting a longitudinal movement of the lead wire with the stoppers, the electrode portion can be more reliably fixed to the pericardium.

In the abovementioned aspect, the stopper may also be provided with a pacing electrode portion for detecting an electrocardiographic signal and outputting a pacing signal.

By so doing, the pacing electrode portion can be fixed to the pericardium separately from the electrode portion of the defibrillation electrode, and thereby the electrocardiographic signal can be more accurately detected to output an appropriate pacing signal.

A fifth aspect of the present invention provides a defibrillator comprising any one of the abovementioned defibrillation electrodes.

A sixth aspect of the present invention provides an endoscope apparatus for use in setting of any one of the abovementioned defibrillation electrodes, comprising: an insertion portion to be inserted into a body; an observation optical system for illuminating illumination light from the distal end of the insertion portion and collecting returning light; and a channel formed throughout along a longitudinal direction of the insertion portion to the distal end surface, for inserting the defibrillation electrode; wherein the distal portion of the insertion portion is provided with a bendable portion which is at least unidirectionally curvable for changing the direction of the distal end surface of the insertion portion, and the circumference of the cross section of the bendable portion comprises an approximate linear portion on the outer side when the bendable portion is curved.

According to the sixth aspect of the present invention, for example, the distal portion of the insertion portion is inserted from the bottom of the xiphoid process into the body. Then, while performing an endoscopic observation by irradiating illumination light on the interior of the body and collecting returning light using the observation optical system, the distal portion of the insertion portion is used to pierce through the pericardium and is placed on the inner surface of the pericardium. In this state, the bendable portion on the distal end of the insertion portion is curved to effect a tight contact between the distal end surface of the insertion portion and the inner surface of the pericardium. Then, the defibrillation electrode is inserted through the channel, by which the lead wire of the defibrillation electrode can be once inserted through the pericardium to place the electrode portion outside the pericardium. At this time, since the movement of the electrode portion is limited by the stopper provided on the lead wire, the electrode portion can be retained in a fixed state to the pericardium.

In this case, when the bendable portion is curved while placing on the heart side the approximate linear portion provided in the cross sectional shape of the bendable portion, the distal end surface of the insertion portion can face toward the inner surface of the pericardium, and thereby can be readily tightly contacted with the inner surface of the pericardium. The outer surface of the heart is curved in an outwardly convex shape. Therefore, if the outer circumferential side of the curvature of the bendable portion is also formed in a convex shape, although the posture of the insertion portion about the axis is unstable, the posture of the insertion portion about the axis can be stabilized by tightly contacting the flat surface formed by providing the approximate linear portion to the outer surface of the convex shape of the heart, and thereby the curving operation can be readily performed.

In the abovementioned aspect, the distal end surface of the insertion portion is preferably inclined oppositely to the linear portion across the axis.

By so doing, even if the angle of curvature of the bendable portion is small, the distal end surface of the insertion portion can be readily placed in a direction along the inner surface of the pericardium.

In addition, in the abovementioned aspect, there may be provided an armored tube which can show up from or retreat behind the distal end surface of the insertion portion through the channel, and can project the defibrillation electrode from the distal opening, and the armored tube may be formed to be oppositely curvable to the bendable portion in a state where the armored tube is being projected from the distal end surface of the insertion portion.

By so doing, when the bendable portion of the insertion portion is curved to effect a tight contact between the distal end surface of the insertion portion and the inner surface of the pericardium and when the armored tube is projected from the channel to pierce through the pericardium, the armored tube is curved oppositely to the direction of curvature of the bendable portion and its distal end faces toward the outer surface of the pericardium. In this state, when the defibrillation electrode is projected from the inside of the armored tube, the pericardium can be again pierced with the defibrillation electrode and the lead wire can be inserted through the pericardium twice, by which the electrode portion placed therebetween can be more reliably fixed to a position along the outer surface of the pericardium.

1. A method of cardiac surgery, comprising: using a device which comprises a treatment unit to be inserted into a thoracic cavity for performing treatment of a heart; inserting a distal end of this device between the heart and a pericardium; thereafter piercing through the pericardium with the distal end of said device; and performing treatment of the heart from the outside of the pericardium by using the treatment unit.

2. A method of cardiac surgery according to claim 1, wherein said device is inserted between said heart and said pericardium by inserting the device from a bottom of a xiphoid process into the thoracic cavity and piercing through said pericardium with the distal end of the device.

3. A method of cardiac surgery according to claim 1, wherein said device is inserted between said heart and said pericardium by inserting the device into a right atrium and piercing through a cardiac wall of a right auricle with the distal end of the device.

4. A method of cardiac surgery according to claim 1, wherein said device is inserted between said heart and said pericardium by inserting the device from the interior of a right atrium into a vein communicated to the interior of the right atrium and located on the cardiac wall, and piercing through the vascular wall of said vein with the distal end of the device.

5. A method of cardiac surgery according to claim 1, wherein said device is inserted between said heart and said pericardium by piercing through a vascular wall of a vein located outside the pericardium from the interior of the vein, and then piercing through said pericardium, with the distal end of the device.

6. A method of cardiac surgery according to claim 1, wherein a cylindrical member into which the device is removably insertable is inserted between said heart and said pericardium, and thereafter said device is inserted between said heart and said pericardium through an interior of the cylindrical member.

7. A method of cardiac surgery according to claim 1, wherein a space for manipulating said treatment unit is retained outside said pericardium by compressively deforming a lung using a balloon placed between said pericardium and the lung.

8. A method of cardiac surgery, comprising: inserting a lead wire of a defibrillator between a pericardium and a heart; thereafter piercing through said pericardium with the distal end of the lead wire; and placing an electrode provided on the distal end of said lead, outside the pericardium.

9. A method of cardiac surgery, comprising: inserting a device for bypassing a coronary artery with use of an internal thoracic artery as a graft, between a heart and a pericardium; thereafter piercing through said pericardium with the distal end of the device to thereby introduce the device to said internal thoracic artery; separating said internal thoracic artery; and connecting the ends of said internal thoracic artery to the coronary artery.

10. A defibrillation electrode, comprising:
an electrode portion to be placed along a surface of a pericardium;
a lead wire which is connected to the electrode portion for piercing through the pericardium; and
a stopper which is provided on the lead wire to be placed in a vicinity of the pierced part of the pericardium in a state where said electrode portion is placed on the surface of the pericardium, and then projected radially outward from the lead wire to limit a longitudinal movement of the lead wire.

11. A defibrillation electrode according to claim 10, wherein said electrode portion comprises an elastic member the external dimension of which is expanded or contracted by its elasticity.

12. A defibrillation electrode according to claim 11, wherein said elastic member consists of a part of the lead wire which is designed to take an approximate linear shape by applying an external force and to take a coiled shape by releasing the external force.

13. A defibrillation electrode according to claim 11, wherein said stopper is fixed to the lead wire by having a space between the stopper and said electrode portion to a degree capable of interposing the pericardium.

14. A defibrillation electrode according to claim 12, wherein
said lead wire is provided on both sides of said electrode portion, two of said stoppers are provided on the lead wire on both sides of said electrode portion, and
the stopper on the distal side comprises a sharp portion which is arranged at the front end to pierce through the pericardium with the lead wire, and a projected portion which is arranged at the rear end to limit a backward insertion through the pericardium.

15. A defibrillation electrode according to claim 10, wherein said stopper is provided with a pacing electrode portion for detecting an electrocardiographic signal and outputting a pacing signal.

16. A defibrillator comprising the defibrillation electrode according to claim 10.

17. An endoscope apparatus for use in setting of the defibrillation electrode according to claim 10, comprising:
an insertion portion to be inserted into a body;
an observation optical system for illuminating illumination light from the distal end of the insertion portion and collecting returning light; and
a channel formed throughout along a longitudinal direction of said insertion portion to the distal end surface, for inserting said defibrillation electrode; wherein
the distal portion of said insertion portion is provided with a bendable portion which is at least unidirectionally curvable for changing the direction of the distal end surface of the insertion portion, and
the circumference of the cross section of the bendable portion comprises an approximate linear portion on the outer side when the bendable portion is curved.

18. An endoscope apparatus according to claim 17, wherein the distal end surface of said insertion portion is inclined oppositely to said linear portion across the axis.

19. An endoscope apparatus according to claim 17, comprising an armored tube which can show up from or retreat behind the distal end surface of said insertion portion through said channel, and can project said defibrillation electrode from the distal opening, wherein
the armored tube is formed to be oppositely curvable to said bendable portion in a state where the armored tube is being projected from the distal end surface of said insertion portion.

20. A method of cardiac surgery according to claim 6, wherein the cylindrical member is inserted from the bottom of the xiphoid process into between said heart and said pericardium, and then its distal end is projected from the pericardium into the thoracic cavity.
